(19) United States
(12) Patent Application Publication
(10) Pub. No.: US 2023/0225994 A1
(43) Pub. Date: Jul. 20, 2023

(54) SUBLINGUAL EPINEPHRINE FORMULATIONS AND METHODS OF USE

(71) Applicant: pHase Pharmaceuticals LLC, Dallas, TX (US)

(72) Inventors: G. Lynn Howard, Dallas, TX (US); Nicholas J. Farina, Solebury, PA (US); Christopher Howard, Dallas, TX (US); Edward J. Walters, Allentown, NJ (US)

(21) Appl. No.: 18/186,918

(22) Filed: Mar. 20, 2023

Related U.S. Application Data
(63) Continuation-in-part of application No. 17/575,489, filed on Jan. 13, 2022.
Application No. 17/575,489 is a division of application No. 16/114,939, filed on Aug. 28, 2018, now Pat. No. 11,253,488.
(60) Provisional application No. 62/554,979, filed on Sep. 6, 2017.

(51) Int. Cl. A61K 31/137; A61K 9/00; A61K 9/20; A61P 3/08; A61K 47/26; A61K 47/38; A61P 37/08; A61K 47/12

(52) U.S. Cl. CPC A61K 31/137; A61K 9/006; A61K 9/2018; A61K 9/2054; A61P 3/08; A61K 47/26; A61K 47/38; A61P 37/08; A61K 47/12

(57) ABSTRACT

The present disclosure provides epinephrine formulations and methods of treating anaphylaxis, methods for concomitant therapy during a cardiac event, methods for treating hypoglycemia, and a prophylactic method for immunotherapy, using the epinephrine formulations disclosed herein. The epinephrine formulations of the present disclosed are formulated for delivery via the oral mucosa. Epinephrine formulations of the present disclosure may further comprise citric acid to improve delivery of epinephrine through the oral mucosa. The epinephrine formulations of the present disclosure induce a robust, global sympathetic response in a subject that is disproportionate to the serum epinephrine concentration in the subject.

### CROSS-REFERENCE TO RELATED APPLICATIONS

This application is a continuation-in-part of U.S. application Ser. No. 17/575,489 filed on Jan. 13, 2022, which is a divisional of U.S. application Ser. No. 16/114,939 filed on Aug. 28, 2018, granted as U.S. Pat. No. 11,253,488, which claims the benefit of U.S. Provisional Application No. 62/554,979 filed on Sep. 6, 2017. The entire contents of these applications are incorporated herein by reference in their entirety.

### BACKGROUND

Anaphylaxis is a Type I hypersensitivity reaction that can be caused by stinging and biting insects, allergen immunotherapy, foods, medications, latex exposure, and exercise. In other cases, anaphylaxis may be idiopathic, and present without an obvious cause. The physiological mechanism of anaphylaxis involves the release of mediators from certain types of white blood cells triggered by either immunologic or non-immunologic mechanisms. Diagnosis of anaphylaxis is based on the presence symptoms following an exposure to a potential anaphylactic trigger. Symptoms include a runny nose, sneezing, coughing, wheezing, chest pain, chest tightness, fainting, dizziness, confusion, weakness, hives, a rash, itchy skin, swollen or red skin, shortness of breath, difficulty breathing, tachycardia, itchy or swollen lips, tongue, or throat, hoarse voice, difficulty swallowing, vomiting, nausea, diarrhea, cramps, weak pulse, paleness, feeling of doom and rapid decrease in blood pressure.

Epinephrine is the primary treatment for anaphylaxis. Urgent administration of epinephrine is critical to prevent progression of anaphylaxis to life-threatening symptoms, such as loss of consciousness, respiratory failure, cardiac arrest, and death. Rapid administration of epinephrine provides the person experiencing anaphylaxis additional time to seek further medical intervention, such as additional epinephrine doses, antihistamines, steroids, and/or intravenous fluids. Epinephrine is an endogenous, sympathomimetic, catecholamine that ameliorates/treats anaphylaxis by activating the sympathetic nervous system, primarily α- and β-adrenergic receptors, which induces vasoconstriction, bronchodilation, glucogenesis, and cardiac stimulation, all of which aid in ending the anaphylactic episode. The successful amelioration/treatment of anaphylaxis has been attributed to the serum concentration of epinephrine provided to a subject, wherein higher levels of serum epinephrine are thought to provide a better outcome in ameliorating/treating anaphylaxis, as higher concentrations of epinephrine are believed to provide more activation of the sympathetic nervous system. Herein, the inventors hypothesize, and report for the first time, that the magnitude of sympathetic activation is not a consequence of serum epinephrine concentration, but rather a consequence of the way in which epinephrine is formulated and delivered to a subject.

Currently, anaphylaxis is temporarily ameliorated/treated by administering epinephrine via intramuscular (IM) or subcutaneous (SC) injection into the thigh muscle of the individual experiencing symptoms of anaphylaxis using an autoinjector, such as an EpiPen®, to induce a high serum concentration of epinephrine in a subject. Autoinjectors, however, may fail to effectively deliver epinephrine systemically into circulation because injection into the muscle relies upon a small window of opportunity for epinephrine to leave the vascular bed and be taken up into circulation. When an individual is experiencing a rapid drop in blood pressure, as is the case during anaphylaxis, the uptake of epinephrine via this route may occur at a greatly diminished capacity or may not occur at all. Thus, researchers have contemplated other epinephrine delivery systems to circumvent these shortcomings, such as ingestible pills or tablets, and inhalers. These alternative methods of delivering epinephrine, however, may not be effective in treating anaphylaxis because the individual experiencing anaphylaxis may not be able to swallow, and airways can be constricted.

The inventors have identified a need to develop a new method of delivering epinephrine to individuals experiencing anaphylaxis because of the deficiencies associated with using conventional autoinjectors and alternative delivery systems of the prior art. To this end, the inventors have contemplated delivering epinephrine through the oral mucosa using sublingual, buccal, or sublabial delivery systems to treat or ameliorate anaphylaxis.

Delivery through the oral mucosa using sublingual, buccal, or sublabial delivery produces a faster onset of action than traditional orally administered tablets because of the well-vascularized nature of the oral mucosa, which increases the portion of compound absorbed through the oral mucosa's blood vessels and bypasses the hepatic first-pass metabolic processes. In addition, compounds administered through the oral mucosa are contained within the oral cavity and are therefore not exposed to the acidic environment of the stomach and gastrointestinal tract, which are well-known to degrade and/or inactivate compounds. Further, the oral mucosa has low enzymatic activity relative to the other tissue implicated in alternative delivery schemes, and therefore the potential for drug inactivation due to biochemical degradation is less rapid and extensive than other administration routes. Additionally, sublingual, buccal, and sublabial delivery systems can be formulated to rapidly dissolve or disintegrate in a subject's mouth without the use of water, which is convenient for the elderly, young children, patients with swallowing difficulties, and in situations where water is not available to the subject. For these specially designed formulations, the small volume of saliva that is available is sufficient to disintegrate or dissolve a tablet in the oral cavity. The drug released from these tablets can be absorbed partially or entirely into the systemic circulation through the oral mucosa. Moreover, the inventors hypothesize that in response to epinephrine, the receptors in the oral mucosa trigger the brain to elicit a robust, global sympathetic response implicated in anaphylactic rescue, via post-synaptic innervation, and may further induce the release of endogenous epinephrine in the subject.

In view of these considerations, the inventors have identified a growing demand to develop novel epinephrine compositions formulated for sublingual, buccal, or sublabial delivery to overcome the deficiencies of the epinephrine delivery systems in the prior art. The inventors provide evidence herein that demonstrates sublingual, buccal, and sublabial epinephrine tablets induce a more robust and global sympathetic response than the delivery systems of the prior art. These results are novel, surprising, and unexpected because the sympathetic response obtained via delivery of epinephrine through the oral mucosa disclosed herein is disproportionate to the serum levels of epinephrine observed in the subject. As such, by providing the epinephrine formulations disclosed herein as pharmaceutical agents, therapeutic benefits can be realized, either individually, collectively, or in conjunction with other pharmaceutical agents.

### SUMMARY

Embodiments of the present disclosure relate to novel sublingual, buccal, and sublabial epinephrine formulations and their use in the amelioration and/or treatment of anaphylaxis or symptoms associated therewith. Embodiments of the present disclosure also relate to methods of treating or ameliorating anaphylaxis.

Embodiments of the present disclosure also provide methods for activating the sympathetic nervous system of a subject, via activation of one or more α-adrenergic receptors and/or activation of one or more β-adrenergic receptors, by administering an epinephrine formulation to the oral mucosa.

These and other features, aspects, and advantages of the present embodiments will become understood with reference to the following description, appended claims, and accompanying figures.

### BRIEF DESCRIPTION OF DRAWINGS

FIG. 1 depicts the maximum serum concentrations (Cmax) of epinephrine observed following administration of an IM injection of epinephrine (USP 0.3 mg/0.3 mL in bitartrate) (n=52) and a sublingual epinephrine tablet (25 mg) (n=60), according to one more embodiments of the disclosure.

FIG. 2 depicts the temporal change in serum glucose levels following the administration of an IM injection of epinephrine (USP 0.3 mg/0.3 mL in bitartrate) (n=52) and a sublingual epinephrine tablet (25 mg) (n=60), according to one more embodiments of the disclosure.

FIG. 3 depicts the temporal change in serum glucose levels normalized by Cmax following the administration of an IM injection of epinephrine (USP 0.3 mg/0.3 mL in bitartrate) (n=52) and a sublingual epinephrine tablet (25 mg) (n=60), according to one more embodiments of the disclosure.

FIG. 4 depicts the temporal change in systolic blood pressure following the administration of an IM injection of epinephrine (USP 0.3 mg/0.3 mL in bitartrate) (n=52) and a sublingual epinephrine tablet (25 mg) (n=60), according to one more embodiments of the disclosure.

FIG. 5 depicts the temporal change in systolic blood pressure normalized by Cmax following the administration of an IM injection of epinephrine (USP 0.3 mg/0.3 mL in bitartrate) (n=52), and a sublingual epinephrine tablet (25 mg) (n=60), according to one more embodiments of the disclosure.

FIG. 6 depicts the maximum serum concentrations (Cmax) of epinephrine observed following administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 7 depicts the temporal change in serum glucose levels following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 8 depicts the temporal change in serum glucose levels normalized by Cmax following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 9 depicts the temporal change in systolic blood pressure following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 10 depicts the temporal change in systolic blood pressure normalized by Cmax following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 11 depicts the temporal change in heart rate following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

FIG. 12 depicts the temporal change in heart rate normalized by Cmax following the administration of an epinephrine auto-injector (“EpiPen”— Epinephrine Injection, USP Auto-Injector, 0.3 mg (Mylan Specialty L.P.)) (n=100) and a sublingual epinephrine tablet (25 mg) (n=100), according to one more embodiments of the disclosure.

### DETAILED DESCRIPTION

Some embodiments provide a formulation comprising an effective amount epinephrine or a salt thereof formulated as an epinephrine formulation. In certain embodiments, a salt of epinephrine may be selected from the group consisting of acetate, ascorbate, bitartrate, borate, hydrochloride, maleate, and sulfate. In certain embodiments, the epinephrine or salt thereof may comprise entirely D enantiomer of epinephrine, entirely L enantiomer of epinephrine, or a mixture or D and L enantiomers of epinephrine. In some embodiments, an epinephrine formulation can be formulated to have varying amounts of epinephrine, salts thereof, enantiomers thereof, or combinations of the foregoing.

In some embodiments, an epinephrine formulation, as described herein, may be formulated such that an effective amount of epinephrine in the formulation may be about 1 mg to about 50 mg. For example the effective amount of epinephrine may be about 1 mg, about 2.5 mg, about 5 mg, about 7.5 mg, about 10 mg, about 12.5 mg, about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, or any range or amount in between any two of the preceding values and any other ranges or amounts disclosed herein.

In certain embodiments, an epinephrine formulation, as described herein, may further comprise an effective amount of citric acid.

In some embodiments, an epinephrine formulation, as described herein, may be formulated such that an effective amount of citric acid in the formulation may be about 0.5 mg to about 5 mg. For example, the effective amount of citric acid may be about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.0 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.8 mg, about 2.81 mg, about 2.82 mg, about 2.83 mg, about 2.84 mg, about 2.85 mg, about 2.86 mg, about 2.87 mg, about 2.88 mg, about 2.89 mg, about 2.90 mg, about 2.95 mg, about 3.0 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.0 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, 5.0 mg, or more, or any range or amount in between any two of the preceding values and any other ranges or amounts disclosed herein.

In some embodiments, the epinephrine formulation may comprise an effective amount of a citrate salt in addition to or in place of citric acid. In such embodiments, the citrate salt may be selected from the group consisting of monosodium citrate, disodium citrate, trisodium citrate, potassium citrate, monocalcium citrate, dicalcium citrate, magnesium citrate, any hydrate thereof, and the like, and any combination thereof. In certain embodiments, an epinephrine formulation can be formulated to have varying amounts of citric acid, citrate salts, or combinations thereof.

In certain embodiments, when an epinephrine formulation is said to comprise an effective amount of a citrate salt in addition to or in place of citric acid, that effective amount of citrate salt in the formulation may be about 0.5 mg to about 5 mg. For example, the effective amount of citrate salt may be about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.0 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.8 mg, about 2.81 mg, about 2.82 mg, about 2.83 mg, about 2.84 mg, about 2.85 mg, about 2.86 mg, about 2.87 mg, about 2.88 mg, about 2.89 mg, about 2.90 mg, about 2.95 mg, about 3.0 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.0 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, 5.0 mg, or more, or any range or amount in between any two of the preceding values and any other ranges or amounts disclosed herein.

In some embodiments, an epinephrine formulation, as described herein, may be formulated for sublingual, buccal, or sublabial administration. To achieve such delivery, an epinephrine formulation for sublingual, buccal, or sublabial administration may be formulated as a suitable delivery vehicle. As used herein, a “suitable delivery vehicle” includes, but is not limited to a tablet, a capsule, a pill, a caplet, a dissolvable oral strip, and the like. In certain embodiments, a suitable delivery vehicle, as described herein, may be formulated as a pharmaceutical version thereof, for example, as a pharmaceutical tablet. In some embodiments, suitable delivery vehicles or pharmaceutical versions thereof, as described herein, may comprise one or more excipients or pharmaceutically acceptable excipients selected from the group consisting of diluents, binders, disintegrants, glidants, lubricants, colorants, flavorants and/or fragrance, sweeteners, coating materials, and any combination thereof.

As described herein, diluents may be comprise one or more of microcrystalline cellulose, such as Avicel® PH-102, Avicel® PH-105, Ceolus™ KG1000, powdered cellulose, anhydrous lactose, lactose monohydrate, spray-dried lactose, mannitol, starch, pregelatinized starch, sorbitol, sucrose, compressible sugar, confectioner's sugar, dextrates, dextrin, dextrose, dibasic calcium phosphate, calcium carbonate, maltose, maltodextrin, kaolin, tribasic calcium phosphate, calcium sulfate, cellaburate, calcium lactate, cellulose acetate, corn syrup, erythritol, ethylcellulose, ethyl acrylate/methyl methacrylate copolymer, fructose, isomalt, alpha-lactalbumin, lactitol, magnesium carbonate, magnesium oxide, methacrylic acid/ethyl acrylate copolymer, methacrylic acid/methyl methacrylate copolymer, polydextrose, sodium chloride, simethicone, pullulan, talc, amino methacrylate copolymer, trehalose, and xylitol.

As described herein, binders may comprise one or more of polyvinylpyrrolidone, such Kollidon® 30 or Povidone K30, copovidone, polyvinyl acetate, D-mannitol, insoluble polyvinylpyrrolidone, crosslinked polyvinylpyrrolidone, carbomer, corn starch, pregelatinized starch, carboxymethylcellulose sodium, hypromellose/hydroxypropyl methylcellulose, polyethylene glycol, hydroxyethyl cellulose, hydroxypropyl cellulose, hydroxyethylmethyl cellulose, calcium carboxymethylcellulose/calcium cellulose glycolate/carmellosum calcium, guar galactomannan/guar gum, ethylcellulose, chitosan, dextrin, hydroxypropyl starch, Ceratonia, inulin, magnesium aluminum silicate, maltodextrin, methylcellulose, dextrates, polyethylene oxide, sodium alginate, starch, liquid glucose, sucrose, compressible sugar, zein, gelatin, polymethacrylates, sorbitol, glucose, and acacia.

As described herein, disintegrants may comprise one or more of crospovidone, crospovidonum, croscarmellose sodium, Ludiflash®, low-substituted hydroxypropyl celluloses, cross-linked celluloses, cross-linked sodium carboxymethyl celluloses, cross-linked carboxymethyl celluloses, cross-linked croscarmelloses, cross-linked starches, sodium starch glycolate, chitosan hydrochloride, corn starch, calcium alginate, calcium sodium alginate, docusate sodium, microcrystalline cellulose, hydroxypropyl starch, magnesium aluminum silicate, methylcellulose, sodium alginate, starch, pregelatinized starch, calcium carboxymethylcellulose/calcium cellulose glycolate/carmellosum calcium, sodium starch glycolate, and powdered cellulose.

As described herein, glidants may comprise one or more of colloidal silicon dioxide, talc, tribasic calcium phosphate, calcium silicate, powdered cellulose, magnesium oxide, sodium stearate, magnesium silicate, silica, magnesium trisilicate, and hydrophobic colloidal silica.

As described herein, lubricants may comprise one or more of magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycols, polysorbates, polyoxyl oleyl ether, polyoxyl hydroxystearate, polyoxyl cetostearyl ether, polyoxyl stearate, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, and talc.

As described herein, colorants may comprise one or more of caramel, ferric oxide, titanium dioxide, ferrosoferric oxide, aluminum oxide, FD & C Red #40/Allura Red AC, amaranth, FD & C Blue #1/Brilliant Blue FCF, canthaxanthin, carmine, carmoisine, curcumin, FD & C Red #3/Erythrosine, Fast Green FCF, Green S, D & C Red #30/Helendon Pink, FD & C Blue #2/Indigo Carmine, Iron Oxide Black, Iron Oxide Red, D & C Red #7/Lithol Rubin BK, Patent Blue V, D & C Red #28/Phloxine B, Iron Oxide Yellow, D & C Red #27/Phloxine O, Ponceau 4R, Quinoline Yellow WS, D & C Yellow #10, riboflavin, FD & C Yellow #5/Tartrazine, and FD & C Yellow #6/Sunset Yellow FCF.

As described herein, flavorants and/or fragrances may comprise one or more of vanillin, peppermint flavor powder, berry flavor powder, strawberry flavor powder, orange flavor powder, lemon flavor powder, orange essence, ethyl maltol, eucalyptus oil, isobutyl alcohol, sodium succinate, adipic acid, almond oil, anethole, benzaldehyde, denatonium benzoate, ethyl acetate, ethyl vanillin, ethylcellulose, fructose, fumaric acid, L-glutamic acid, lactitol, leucine, malic acid, maltol, menthol, racementhol, methionine, methyl salicylate, monosodium glutamate, peppermint oil, peppermint spirit, racemethionine, rose oil, rose water, sodium acetate, sodium lactate, tartaric acid, thymol, inulin, isomalt, and neohesperidin dihydrochalcone.

As described herein, sweeteners may comprise one or more of sucralose, saccharin sodium, neotame, sucrose, acesulfame potassium, aspartame, aspartame acesulfame, corn syrup, corn syrup solids, dextrates, dextrose, erythritol, fructose, galactose, glucose, glycerin, inulin, invert sugar, isomalt, lactitol, maltitol, maltose, mannitol, saccharin, saccharin calcium, sorbitol, starch hydrolysate, compressible sugar, confectioner's sugar, tagatose, trehalose, and xylitol.

As described herein, coating materials may comprise one or more of sodium carboxymethylcellulose, cellulose acetate, cellulose acetate phthalate, ethylcellulose, polylactic acid, polyglycolic acid, regenerated collagen, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein, polyesters, polyamino acids, polyvinylpyrrolidone, ethylenevinyl acetate, methylcellulose, and protamine sulfate.

The methods of preparing suitable delivery vehicles, as described herein, are not particularly limited. In some instances, conventional tablet processing methods may be employed. In certain embodiments, the manufacturing process involves granulating low-moldable sugars (e.g., mannitol, lactose, glucose, sucrose, and erythritol) with high-moldable sugars (e.g., maltose, sorbitol, trehalose, and maltitol). The epinephrine or salt thereof can be added, along with other citric acid and/or a salt thereof, excipients or pharmaceutically acceptable excipients, as described herein, during granulation or blending processes. The tablets are manufactured at a low compression force followed by an optional humidity conditioning treatment to increase tablet hardness. In other instances, the manufacturing process involves the direct compression of epinephrine or a salt thereof, citric acid and/or a salt thereof, and excipients or pharmaceutically acceptable excipients, whereby the citric acid and excipients are mixed with the epinephrine or salt thereof and then compressed into tablets. Tablets made via the aforementioned methods may have an adequate hardness for packaging in bottles and easy handling. Tablets made via the methods disclosed herein may also have highly moldable characteristics and fast-dissolving properties.

While not wishing to be bound to a specific hypothesis, it is believed that delivery of epinephrine through the oral mucosa using sublingual, buccal, or sublabial delivery confers superior benefits compared to conventional subcutaneous/intramuscular injection-based delivery methods. During anaphylaxis, the blood pressure of a subject drops, and the subject can become hypotensive, wherein blood flow is directed away from the extremities to the head and organs in the trunk. In this scenario, intramuscular/subcutaneous injection of epinephrine may be insufficient because there is no mechanism to transport the epinephrine from the site of injection to the systemic blood supply, owing to decreased and/or inadequate blood pressure. Even without experiencing anaphylaxis, this issue may exist for subjects with low blood pressure, who may be unable to absorb epinephrine administered by intramuscular injection. Administration of epinephrine through the oral mucosa, using the delivery systems described herein, overcomes these problems. Sublingual, buccal, or sublabial tablets, as described herein, can be administered to subjects experiencing anaphylaxis, even if they have low blood pressure. Moreover, it is believed that administering epinephrine through the oral mucosa activates the receptors therein, thereby triggering the brain to elicit a robust, global sympathetic response via post-synaptic innervation, and may further induce the production and/or release of endogenous epinephrine in the subject. The magnitude of this sympathetic response can be disproportionate to the serum epinephrine levels of the subject provided by the epinephrine delivery system. Because (i) sympathetic response is required for anaphylactic rescue, (ii) sympathetic activation can induce production and/or release of endogenous epinephrine, which can further aide in anaphylactic rescue, and (iii) the epinephrine formulations disclosed herein rely heavily on this mechanism of action, the epinephrine formulations disclosed herein can provide a more robust treatment or amelioration of anaphylaxis than conventional epinephrine delivery systems of the prior art, which rely primarily on providing a high serum concentration of epinephrine.

While not wishing to be bound to a specific hypothesis, it is believed that the citric acid may improve the diffusion of the epinephrine into epithelial cells of the oral mucosa. This improved diffusion may result in increased epinephrine being available for longer, in effect, creating a reservoir of epinephrine that can be beneficial to the subject. It is also theorized that the citric acid increases the amount of epinephrine absorbed and increases duration of absorption. For example, the citric acid allows rapid initial absorption, while also creating an epinephrine reservoir in the oral mucosa. This reservoir creates a longer duration of active absorption. As the circulating levels of epinephrine dissipate, the epinephrine saturating the oral mucosa, through diffusion, enters the subject's bloodstream and continues to activate the sympathetic nervous system, allowing for longer periods of elevated plasma epinephrine levels, a longer time to reach maximum epinephrine serum levels, and a longer duration of physiological responses critical for anaphylactic rescue resulting from sympathetic activation. Thus, when citric acid is included in an epinephrine formulation, as described herein, the citric acid is not an excipient, as it directly impacts the effectiveness/outcome of the epinephrine formulation as a therapeutic treatment.

In some embodiments, a suitable delivery vehicle, as described herein, may be formulated as a rapid dissolution or a fast dissolution vehicle. As used herein, “rapid dissolution” or “fast dissolution vehicle,” generally refers to suitable delivery vehicles that rapidly dissolve within the buccal, sublingual, or sublabial cavities. Rapid/fast dissolution, in this context, refers to full disintegration of a tablet within about 200 seconds. For example, rapid/fast dissolution may refer to full disintegration of a tablet within about 120 seconds, about 100 seconds, about 80 seconds, about 60 seconds, about 50 seconds, about 40 seconds, about 30 seconds, about 20 seconds, or any range or amount in between any two of the preceding values and any other ranges or amounts disclosed herein.

In certain embodiments, suitable delivery vehicles, as described herein, may be formulated to have a desired dissolution time. In some embodiments, the desired dissolution time of the tablet may be about 12% to about 100% dissolution within 5 minutes. In other embodiments, the desired dissolution time of the tablet may be about 35% to about 100% dissolution within 5 minutes. In some embodiments, the desired dissolution time of the tablet may be about 50% to about 100% dissolution within 5 minutes. In certain embodiments, the desired dissolution time of the tablet may be about 75% to about 100% dissolution within 5 minutes. In some embodiments, the desired dissolution time of the tablet may be about 45% to about 100% dissolution within 10 minutes. In other embodiments, the desired dissolution time of the tablet may be about 50% to about 100% dissolution within 10 minutes. In certain embodiments, the desired dissolution time of the tablet may be about 90% to about 100% dissolution within 10 minutes. In some embodiments, the desired dissolution time of the tablet may be about 95% to about 100% dissolution within 15 minutes to 30 minutes. It will be understood that variations in dissolution times may occur based on manufacturing methodology.

Some embodiments provide an epinephrine formulation, as described herein, as a prophylactic treatment for immunotherapy. Immunotherapy is a prolonged and expensive treatment; however, in some instances, it is the only viable remedy for allergies. A concern with immunotherapy is the potential to cause severe or even fatal systemic reactions. Using an epinephrine formulation, as described herein, concurrently with allergen injections can not only diminish that concern, but also may allow a higher allergen concentration of exposure to potentially shorten the course of immunotherapy and provide allergen protection sooner. Because allergen immunotherapy introduces an allergen into an allergic individual, hypersensitivity reactions are often unavoidable. Epinephrine formulations, as described herein, can mitigate risk and can be effective prophylactic and therapeutic treatments. Due to a fast onset of action and a robust induced response, a method of using an epinephrine formulation, as described herein, would require that the patient receiving immunotherapy place an epinephrine formulation, as described herein, in the oral cavity, i.e., sublingually, buccally, or sublabially, at the first sign of any adverse event resulting from an immunotherapy injection of pre-determined allergen. Administration of an epinephrine formulation, as described herein, can block the cascade resulting in anaphylaxis before it begins and potentially mitigate biphasic anaphylaxis due to its sustained epinephrine action, through pronged elevated blood serum levels, prolonged sympathetic activation, or both.

Some embodiments provide an epinephrine formulation, as described herein, for concomitant use during cardio-pulmonary resuscitation (CPR). Epinephrine is the primary drug administered during CPR to reverse cardiac arrest. Epinephrine increases arterial blood pressure and coronary perfusion during CPR via agonistic action on the α-1 adrenergic receptor, which increases myocardial and cerebral blood flow. Clinical data suggests that epinephrine increases a short-term return of pulse during life-threatening cardiac event. Once spontaneous circulation is restored, α- and/or β-adrenergic agonists may be needed for circulatory support once hospitalized/stable. Because the current epinephrine-containing therapies are invasive, require specialized training to administer, and are expensive to acquire, store, and replace, it is currently not a viable concomitant treatment with CPR for the average bystander or small business. Epinephrine formulations, as described herein, bypass these shortcomings as they are less costly, and easier to use, administer, and store. Once it is determined that CPR may be administered, prior to chest compressions, a method of using an epinephrine, as described herein, immediately provides a dose of epinephrine, by placing said formulation in the oral cavity of the subject, i.e., sublingually, buccally, or sublabially, before proceeding with chest compressions. The epinephrine formulations of the present disclosure may enter the blood stream rapidly, and induce a robust sympathetic response, thereby returning a pulse to the subject quickly, which could be helpful to the one administering CPR, particularly if the person is performing compressions alone and may fatigue before the patient stabilizes.

Some embodiments provide an epinephrine formulation, as described herein, for single use in treating or preventing hypoglycemia in diabetic subjects. Hypoglycemia is a condition characterized by abnormally low blood glucose levels—usually less than 70 mg/dL. Fear of a severe hypoglycemic reaction is a major obstacle to achieving near-normal plasma glucose levels. Although parenteral glucagon is effective in treating these reactions, it is cumbersome to use, causes severe nausea, and is impractical in the home or school setting. Alternatively, the use of epinephrine formulations, as described herein, may be used by all care providers, be it a clinical or non-clinical setting. Embodiments disclosed herein induce a robust glucose response and may therefore be effective at treating hypoglycemia. Embodiments of the disclosure are safe, and easy-to-use alternative to glucagon, particularly in children. Moreover, epinephrine formulations, as disclosed herein, are low cost, stable, and easy-to-use, requiring no specialized training to administer as a therapeutic. An epinephrine formulation of the present disclosure may be administered as a single dose at the onset of hypoglycemia or early symptoms associated therewith, including, but not limited to sweating, dizziness, palpitations, tremors, hunger, restlessness, tingling in the hands, feet, lips, or tongue, lightheadedness, inability to concentrate, headache, drowsiness, difficulty sleeping, anxiety, blurred vision, slurred speech, depressed mood, abnormal behavior, unsteady movement, personality changes, and any combination of the foregoing. If not treated, the symptoms may progress to severe hypoglycemia, with disorientation, unconsciousness, seizures, and potentially death.

Some embodiments provide an epinephrine formulation, as described herein, to induce a sympathetic response within an individual. The sympathetic response may be disproportionate to that which would reasonably be expected from the levels of epinephrine circulating in the blood stream. In some instances, the sympathetic response may arise through agonistic action on an α-adrenergic receptor, a β-adrenergic receptor, or both. In some embodiments, a sympathetic response may manifest in a subject as one or more selected from the group consisting of a metabolic response, a gastrointestinal response, an endocrine response, a cardiovascular response, a neurological response, and an immunological response.

As used herein, a “metabolic response” is characterized by a subject exhibiting an increase in blood glucose levels.

As used herein, a “gastrointestinal response” is characterized by a subject exhibiting one or more of the following: abdominal pain/cramping/discomfort, burping, itching/irritation/soreness of the throat, heartburn, stomachache, nausea, vomiting, dry heaving, diarrhea, jaw pain, dry mouth, and hiccups.

As used herein, an “endocrine response” is characterized by a subject exhibiting hyperhidrosis.

As used herein, a “cardiovascular response” is characterized by a subject exhibiting one or more of the following: an increase in systolic blood pressure, heart racing, flushing, chest pain, abnormal electrocardiogram, dizziness, shakiness, shortness of breath, hyperthermia, and chills.

As used herein, a “neurological response” is characterized by a subject exhibiting one or more of the following: headache, light headedness, drowsiness, jitteriness, numbness, and pruritus.

As used herein, an “immunological response” is characterized by a subject exhibiting an upregulation of neutrophils.

By way of example, a sympathetic response, as described herein, may be a change in blood glucose levels; specifically, an increase in blood glucose levels. In certain embodiments, an increase in blood glucose can be effectuated in a subject following the administration of an epinephrine formulation, as described herein. The increase in blood glucose levels may be effectuated in the subject for at least about 3 minutes following administration of the epinephrine formulation. The duration of the increase in blood glucose levels may be for at least about 240 minutes following the administration of the epinephrine formulation. For example, the duration of increase in blood glucose levels may be at least about 3 minutes, about 6 minutes, about 10 minutes, about 15 minutes, about 20 minutes, about 30 minutes, about 45 minutes, about 60 minutes, about 90 minutes, about 120 minutes, about 240 minutes, or longer, or any range or time in between any two of the preceding values and any other ranges or times disclosed herein. Notably, the increase in blood glucose levels can be disproportionate to the serum epinephrine levels. To accurately describe the increase in blood glucose levels, as described herein, a “normalized” increase in blood glucose levels should be adopted to account for the levels of epinephrine in a subject's blood; wherein a “normalized” increase in blood glucose levels is determined by dividing the increase in blood glucose level for a particular time point by the maximum epinephrine serum level observed. In some embodiments, an epinephrine formulation, as described herein, provides a normalized increase in blood glucose levels that is higher than any known epinephrine delivery system of the prior art. The epinephrine formulations disclosed herein may provide a normalized increase in blood glucose levels that is higher than that of an intramuscular or subcutaneous epinephrine injection at about 3 minutes after administration, at about 6 minutes after administration, at about 10 minutes after administration, at about 15 minutes after administration, at about 20 minutes after administration, at about 30 minutes after administration, at about 45 minutes after administration, at about 60 minutes after administration, at about 90 minutes after administration, at about 120 minutes after administration, at about 240 minutes after administration, or at a later time point, or any range or time in between any two of the preceding values and any other times or amounts disclosed herein.

By way of example, a sympathetic response, as described herein, may be a change in systolic blood pressure; specifically, an increase in systolic blood pressure. In certain embodiments, an increase in systolic blood pressure can be effectuated in a subject following the administration of an epinephrine formulation, as described herein. The increase in systolic blood pressure may be effectuated in the subject for at least about 3 minutes following administration of the epinephrine formulation. The duration of the increase in systolic blood pressure may be at least about 120 minutes following the administration of the epinephrine formulation. For example, the duration of increase in systolic blood pressure may be for at least about 3 minutes, about 6 minutes, about 10 minutes, about 15 minutes, about 20 minutes, about 30 minutes, about 45 minutes, about 60 minutes, about 90 minutes, about 120 minutes, or any range or time in between any two of the preceding values and any other ranges or times disclosed herein. Notably, the increase in systolic blood pressure can be disproportionate to the serum epinephrine levels. To accurately describe the increase in systolic blood pressure, as described herein, a “normalized” increase in systolic blood pressure should be adopted to account for the levels of epinephrine in a subject's blood; wherein a “normalized” increase in systolic blood pressure is determined by dividing the increase in systolic blood pressure for a particular time point by the maximum epinephrine serum level effectuated. In some embodiments, an epinephrine formulation, as described herein, provides a normalized increase in systolic blood pressure that is higher than any known epinephrine delivery system of the prior art. The epinephrine formulations disclosed herein may provide a normalized increase in systolic blood pressure that is higher than that of an intramuscular or subcutaneous epinephrine injection at about 3 minutes after administration, at about 6 minutes after administration, at about 10 minutes after administration, at about 15 minutes after administration, at about 20 minutes after administration, at about 30 minutes after administration, at about 45 minutes after administration, at about 60 minutes after administration, at about 90 minutes after administration, at about 120 minutes after administration, or at a later time point, or any range or time in between any two of the preceding values and any other ranges or times disclosed herein.

By way of example, a sympathetic response, as described herein, may be a change in heart rate from baseline; specifically, an increase in heart rate. In certain embodiments, an increase in heart rate can be effectuated in a subject following the administration of an epinephrine formulation, as described herein. The increase in heart rate may be effectuated in the subject for at least about 3 minutes following administration of the epinephrine formulation. The duration of the increase in heart rate may be for at least about 120 minutes following the administration of the epinephrine formulation. For example, the duration of increase in heart rate may be for at least about 3 minutes, about 6 minutes, about 10 minutes, about 15 minutes, about 20 minutes, about 30 minutes, about 45 minutes, about 60 minutes, about 90 minutes, about 120 minutes, or any range or time in between any two of the preceding values and any other ranges or times disclosed herein. Notably, the increase in heart rate can be disproportionate to the serum epinephrine levels. To accurately describe the increase in heart rate, as described herein, a “normalized” increase in heart rate should be adopted to account for the levels of epinephrine in a subject's blood; wherein a “normalized” increase in heart rate is determined by dividing the increase in heart rate for a particular time point by the maximum epinephrine serum level effectuated. In some embodiments, an epinephrine formulation, as described herein, provides a normalized increase in heart rate that is higher than any known epinephrine delivery system of the prior art. The epinephrine formulations disclosed herein may provide a normalized increase in heart rate that is higher than that of an intramuscular or subcutaneous epinephrine injection at about 3 minutes after administration, at about 6 minutes after administration, at about 10 minutes after administration, at about 15 minutes after administration, at about 20 minutes after administration, at about 30 minutes after administration, at about 45 minutes after administration, at about 60 minutes after administration, at about 90 minutes after administration, at about 120 minutes after administration, or at a later time point, or any range or time in between any two of the preceding values and any other ranges or times disclosed herein.

By way of example, a sympathetic response, as described herein, may be an occurrence of a physiological response associated with epinephrine, other than those listed above, in a subject population; specifically an increase in frequency of occurrence of a physiological response associated with epinephrine in a subject population. As used herein, a “physiological response associated with epinephrine” may refer to any of the following: abdominal pain/cramping/discomfort, burping, itching/irritation/soreness of the throat, heartburn, stomachache, nausea, vomiting, dry heaving, diarrhea, jaw pain, dry mouth, hiccups, hyperhidrosis, heart racing, flushing, chest pain, abnormal electrocardiogram, dizziness, shakiness, shortness of breath, hyperthermia, chills, headache, light headedness, drowsiness, jitteriness, numbness, pruritus, upregulation of neutrophils, and the like. In some embodiments, the frequency of occurrence of a physiological response associated with epinephrine is observed in a subject population following the administration of an epinephrine formulation may be at least about 60% of the subject population. For example, the frequency of occurrence of a physiological response associated with epinephrine may be at least about 60% of a subject population receiving an epinephrine formulation described herein, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, or any range or amount in between any two of the preceding values and any other ranges or amounts disclosed herein. In certain embodiments, an increase in frequency of occurrence of a physiological response associated with epinephrine can be observed in a subject following the administration of an epinephrine formulation, as described herein, compared to known epinephrine delivery systems of the prior art. The magnitude of the increase in frequency of occurrence of a physiological response associated with epinephrine resulting from the epinephrine formulations disclosed herein may be at least 4-fold higher than known epinephrine delivery systems of the prior art. For example, the magnitude of increase may be at least 4-fold higher, 5-fold higher, 6-fold higher, 7-fold higher, 8-fold higher, 9-fold higher, 10-fold higher, or more, or any range or amount in between any two of the preceding values and any other ranges or amounts disclosed herein. Notably, the increase in frequency of occurrence of a physiological response associated with epinephrine is disproportionate to the serum epinephrine levels. To accurately describe the increase in frequency of occurrence of a physiological response associated with epinephrine, as described herein, a “normalized” increase in frequency of occurrence of a physiological response associated with epinephrine should be adopted to account for the levels of epinephrine in a subject's blood; wherein a “normalized” increase in frequency of occurrence of a physiological response associated with epinephrine is determined by dividing the increase in frequency of occurrence of a physiological response associated with epinephrine by the maximum epinephrine serum level observed. In some embodiments, a normalized increase in frequency of occurrence of a physiological response associated with epinephrine can be effectuated in a subject following the administration of an epinephrine formulation, as described herein, compared to known epinephrine delivery systems of the prior art. The magnitude of the normalized increase in frequency of occurrence of a physiological response associated with epinephrine resulting from the epinephrine formulations disclosed herein may be at least 8-fold higher than known epinephrine delivery systems of the prior art. For example, the magnitude of increase may be at least 8-fold higher, 10-fold higher, 12-fold higher, 14-fold higher, 16-fold higher, 18-fold higher, 20-fold higher, 22-fold higher, 24-fold higher, 26-fold higher, 28-fold higher, 30-fold higher, 32-fold higher, 34-fold higher, 36-fold higher, or more, or any range or amount in between any two of the preceding values and any other ranges or amounts disclosed herein.

As used herein, “identifying,” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, the terms “prophylactic,” “prevent,” or “preventing,” and the like can refer to treating a subject who does not yet exhibit symptoms of a disease or condition, but who is susceptible to, or otherwise at risk of, a particular disease or condition, whereby the treatment reduces the likelihood that the patient will develop the disease or condition. A “disorder” is any condition that would benefit from treatment with the compositions described herein.

As used herein, the terms “preventing”, “treating”, “treatment” and the like are used herein to generally refer to obtaining a desired pharmacological and physiological effect which will be clear to the skilled artisan based upon the context in which these terms are used. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein encompasses any treatment of a disease in a mammal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms, conditions, and co-morbidities. The terms “optimum” or “healthy” and the like may be used to refer to the baseline physiological state of a mammal, wherein administration of sublingual epinephrine formulations, as described herein, may be administered to a mammal that may be experiencing a disease/condition or symptoms thereof that causes a deviation from baseline, such as anaphylaxis, hypoglycemia, cardiac arrest, suppressed sympathetic response, and the like. In this case, epinephrine formulations, as described herein, may be administered to return the physiological state of a subject to its “healthy” or “optimum” state—that is—the physiological state prior to experiencing any of the aforementioned diseases/conditions or symptoms thereof.

As used herein, the phrase “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements. For example, the use of a composition “consisting essentially of a composition” for the treatment of a particular disease or disorder, or the maintenance of a healthy condition, would exclude other ingredients that would materially alter the intended outcome of the composition.

As used herein, a composition that “substantially” comprises a compound means that the composition contains more than about 80% by weight, more preferably more than about 90% by weight, even more preferably more than about 95% by weight, and most preferably more than about 98% by weight of the compound.

The term “pharmaceutical formulation”, “formulation”, “composition” and the like can refer to preparations which are in such a form as to permit the biological activity of the active ingredients to be effective, and therefore may be administered to a subject for therapeutic use. The meaning of these terms will be clear to the skilled artisan based upon the context in which they are used.

A “therapeutically effective amount” as used herein includes within its meaning a non-toxic but sufficient amount of a compound active ingredient or composition comprising the same for use in the embodiments disclosed herein to provide the desired therapeutic effect. Similarly, “an amount effective to” or “an effective amount” as used herein includes within its meaning a non-toxic but sufficient amount of a compound active ingredient or composition comprising the same to provide the desired effect. A “therapeutically effective amount” or an “effective amount” includes amounts of compounds that would not be achievable through a standard diet, but requires supplementation and dosing, as described herein. The exact amount of the active ingredient disclosed herein required will vary from subject to subject depending on factors such as the species being treated, the age and general condition of the subject, the severity of the condition being treated, the particular agent being administered, the weight of the subject, and the mode of administration and so forth. Thus, it may not always be possible to specify an exact “effective amount.” However, for any given case, an appropriate “effective amount” may be determined by one of ordinary skill in the art in view of the disclosure contained herein. In some aspects, a therapeutically effective amount may include a dosing regimen.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In addition, the appropriate dosage of the compositions can depend, for example, on the condition to be treated, the severity and course of the condition, whether the composition is administered for preventive or therapeutic purposes, previous therapy, the patient's clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

While exemplary embodiments, aspects and variations have been provided herein, those of skill in the art will recognize certain modifications, permutations, additions and combinations and certain sub-combinations of the embodiments, aspects, and variations. It is intended that the following claims are interpreted to include all such modifications, permutations, additions and combinations and certain sub-combinations of the embodiments, aspects and variations are within their scope.

### EXAMPLES

### Example 1

Sublingual epinephrine tablets, as described herein, were produced with an effective dose of 25 mg of L-epinephrine. To achieve this dose, 45.5 mg of L-epinephrine bitartrate was hand-mixed with citric acid and various excipients in distilled water. The tablets were compressed and both tables produced were generally round and did not crumble. Two 25 mg sublingual epinephrine tablets, according to the disclosure, were made and are shown below in Tables 1 and 2.

TABLE 1
A sublingual epinephrine tablet formulation,
according to the disclosure

| | Formulation 1 - 201 study | | |
|---|---|---|---|
| Component | Concentration (% w/w) | Mass (mg) | |
| Epinephrine bitartrate | 20.22% | 45.5 | mg* |
| Avicel PH 102 | 12.75% | 28.69 | mg |
| Avicel PH 105 | 3.36% | 7.56 | mg |
| Ludiflash | 56.42% | 126.94 | mg |
| Croscarmellose sodium | 5.00% | 11.25 | mg |
| Citric acid, anhydrous | 1.25% | 2.81 | mg |
| Magnesium stearate | 1.00% | 2.25 | mg |
| Total | 100% | 225 | mg |

*Conversion factor for epinephrine bitartrate to epinephrine is 1.82:1.00; i.e., 45.5 mg of epinephrine bitartrate corresponds to a 25 mg dose of epinephrine.

TABLE 2
A sublingual epinephrine tablet formulation,
according to the disclosure

| | Formulation 2 - 202/203 study | | |
|---|---|---|---|
| Component | Concentration (% w/w) | Mass (mg) | |
| Epinephrine bitartrate | 47.89% | 45.5 | mg* |
| Ceolus KG1000 | 15.00% | 14.25 | mg |
| Povidone K30 | 3.16% | 3.0 | mg |
| Crospovidone | 28.74% | 27.30 | mg |
| Citric acid monohydrate | 4.21% | 4.00 | mg |
| Magnesium stearate | 1.00% | 0.95 | mg |
| Total | 100% | 95 | mg |

*Conversion factor for epinephrine bitartrate to epinephrine is 1.82:1.00; i.e., 45.5 mg of epinephrine bitartrate corresponds to a 25 mg dose of epinephrine.

### Example 2

A sublingual epinephrine tablet (25 mg dose of L-epinephrine) (n=60), as described herein (Formulation 1), was evaluated in a clinical trial against an IM injection of epinephrine (USP 0.3 mg/0.3 mL in bitartrate) (n=52).

Treatments above were administered by two means. Sublingual tablets were administered by placing the tablet under the subject's tongue, as far back as possible. The subject was instructed not to swallow, chew, suck, or bite the tablet. Complete dissolution of the tablet is confirmed by clinical staff. Second, IM injections were administered by clinical staff to the anterolateral aspect of the thigh of the subject, in accordance with CRO-approved instructions.

The clinical trial enrolled a population, with inclusion criteria being healthy, non-tobacco, non-nicotine-using, adult male and female satisfying all entry criteria. Inclusion criteria were males and non-pregnant females 18-45 years of age, inclusive, with a body mass index (BMI) of 20.0-30.0 kg/m², inclusive, where BMI was calculated by Novum Pharmaceutical Research Services Standard Operating Procedure. Further inclusion criteria for women included satisfying at least one of (i)(a) agree to abstain from sexual intercourse from screening and throughout the duration of the study and have used, (b) agree to continue to use a reliable method of contraception (e.g., condom with spermicide, IUD, hormonal contraceptives) for at least 30 days before initial dosing and throughout the duration of the study, (c) surgically sterile (bilateral oophorectomy or hysterectomy, bilateral tubal ligation at least 3 months before initial dosing or Essure® device placement before the year 2018), (d) at least 1 year postmenopausal and have a documented FSH level ≥40 mIU/mL at screening; (ii) judged to be in good health by an Investigator based on medical history, physical examination, vital sign measurements, clinical laboratory tests, and 12-Lead ECG obtained at screening; (iii) serum potassium level within the laboratory's normal reference at screening; and (iv) signed and dated informed consent form, which meets all criteria of current FDA regulations.

Exclusion criteria were any of the following: (i) females who are pregnant, lactating, or likely to become pregnant during the study; (ii) history of allergy, sensitivity to epinephrine, sulfites, or sympathomimetic amines or history of any drug hypersensitivity or intolerance which, in the opinion of the Investigator, would compromise the safety of the subject or the study; (iii) significant history or current evidence of chronic infectious disease, system disorders, organ dysfunction, especially cardiovascular disorders (angina, cardiac arrhythmias, coronary artery or organic heart disease, myocardial infarction, hypertension), glaucoma, seizures, pulmonary edema, diabetes, hypothyroidism or hyperthyroidism, respiratory disorders, or obesity; (iv) clinically significant findings in the 12-lead ECG at screening, at the discretion of the Investigator; (v) seated blood pressure greater than 140/90 mmHg or pulse rate greater than 90 bpm at screening; (vi) any signs or symptoms or history of bronchial asthma or emphysema; (vii) clinically significant history or presence of gastrointestinal disease or history of malabsorption within the last year, as determined by the Investigator; (viii) current piercings in the mouth, including lips and cheek that have studs/rings, etc. or where the piercing wound is not completely closed; (ix) any tongue or other oral deformities that may affect the absorption of the drug products; (x) wears braces, temporary dental caps or any other orthodontic appliance or has had any dental procedures within 2 weeks of dosing or anticipated during the study. Any significant dental issues were noted during the screening oral examination. If dentures are worn, they must be removed during dosing procedures; (xi) history of psychiatric disorders (e.g., depression, psychosis, suicidal thoughts) occurring within the last two years that required the subject to be hospitalized or treated with medication; (xii) presence of a medical condition requiring regular treatment with prescription drugs; (xiii) use of pharmacologic agents known to significantly induce or inhibit drug-metabolizing enzymes within 30 days before initial dosing; (xiv) use of tricyclic antidepressants, monoamine oxidase (MAO) inhibitors, levothyroxine sodium, antihistamines (especially chlorpheniramine, tripelennamine, and diphenhydramine), cardiac glycosides, diuretics, anti-arrhythmics, beta- and alpha-adrenergic blocking drugs, and ergot alkaloids within 14 days before initial dosing; (xv) receipt of any drug as part of a research study within 30 days before initial dosing; (xvi) drug or alcohol addiction requiring treatment during the past 12 months; (xvii) history of excessive alcohol consumption (on average more than 14 units of alcohol/week) during the past 12 months; (xviii) positive test for HIV, Hepatitis B surface antigen or Hepatitis C antibody; (xix) positive test results for drugs of abuse at screening; (xx) if female, has a positive pregnancy test at screening; and (xxi) use of tobacco- or nicotine-containing products within 30 days before initial dosing.

Continuing eligibility were the following: (i) the use of pharmacologic agents known to significantly induce or inhibit drug-metabolizing enzymes within 30 days before initial dosing and throughout the duration of the study is prohibited; (ii) the use of tricyclic antidepressants, monoamine oxidase (MAO) inhibitors, levothyroxine sodium, antihistamines (especially chlorpheniramine, tripelennamine, and diphenhydramine), cardiac glycosides, diuretics, anti-arrhythmics, beta- and alpha-adrenergic blocking drugs, and ergot alkaloids within 14 days before initial dosing and throughout the duration of the study is prohibited; (iii) the use of prescription medications within 14 days before initial dosing and throughout the duration of the study should be avoided; (iv) the use of over-the-counter (OTC) medications (including vitamins and herbal products) or diet supplement products (e.g., Cellucor®, Muscle Tech®) within 72 hours before initial dosing and throughout the duration of the study should be avoided; (v) alcohol- or grapefruit-containing food or beverages (e.g., Fresco®) or energy drinks (e.g., Red Bull, 5-Hour Energy®, Monster Energy®) ingested within 48 hours before initial dosing and throughout the duration of the study should be avoided; (vi) caffeine/xanthine-containing food or beverages (e.g., chocolate, coffee, tea, cola) ingested within 24 hours before initial dosing and throughout the duration of the study should be avoided. Violations of the above restrictions resulted in the Investigator excluding the subject from entering the study or discontinuing the subject's ongoing participation. Individual exceptions to the above restrictions, that the Investigator did not believe affected subject safety or the integrity of the study data, were allowed if approved by Sponsor's Representative. All subjects were screened for alcohol, marijuana (THC) metabolites, amphetamine (AMP), methamphetamines (MET), and cocaine metabolites using immediate saliva/urine testing at check-in. Subjects with positive results were withdrawn from the study. All female subjects had a urine pregnancy test performed at check-in. Subjects with positive results were withdrawn from the study.

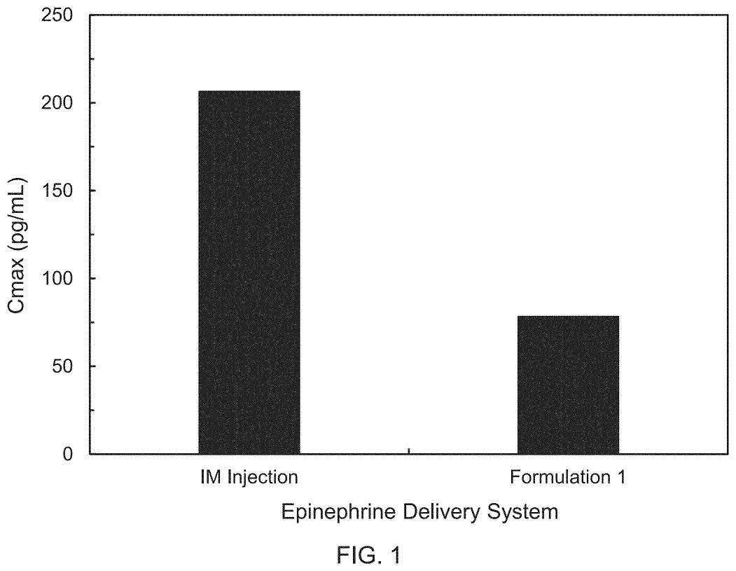

The clinical trial was performed to evaluate the maximum serum concentrations (Cmax) of epinephrine following the administration of the aforementioned treatments; and metrics associated with sympathetic response, which is required for anaphylactic rescue, namely: (i) change in serum glucose levels (at 60 minutes and 240 minutes), (ii) change in systolic blood pressure (at 30 minutes, 60 minutes, and 120 minutes), and (iii) incidence of gastrointestinal or other evidence of sympathetic activation, aside from change in blood glucose levels and systolic blood pressure. The data from the clinical studies are reported in FIGS. 1-5.

FIG. 1 depicts the observed Cmax following the administration of the IM injection of epinephrine (“IM Injection”), described above, and the sublingual epinephrine tablet (“Formulation 1”), described above. FIG. 1 demonstrates that the IM epinephrine injection produced a mean Cmax of about 206 pg/mL, which was significantly larger than mean Cmax of about 78 pg/mL observed for the sublingual epinephrine tablets of Formulation 1. This result was unexpected, as the sublingual epinephrine tablet was expected to have a similar or larger maximum serum concentration of epinephrine compared to an IM injection.

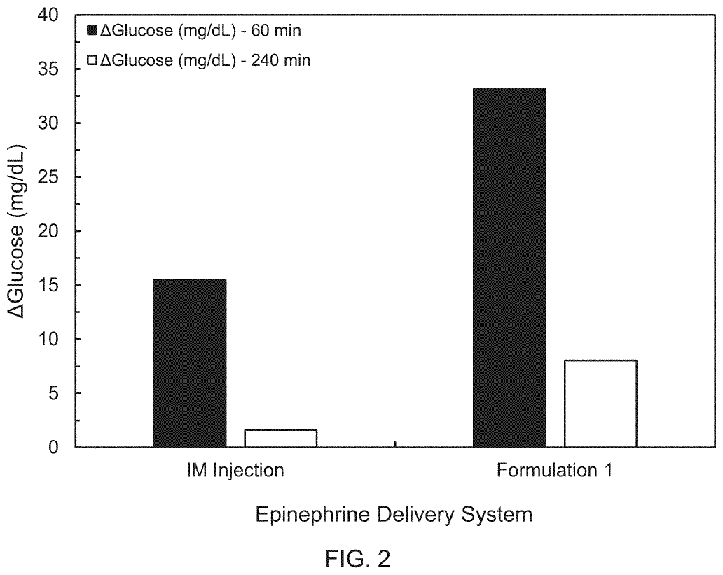

FIG. 2 depicts the temporal change in serum glucose levels at 60 minutes (black) and 240 minutes (white) following the administration of the IM injection of epinephrine (“IM Injection”), described above, and the sublingual epinephrine tablet (“Formulation 1”), described above. The results of FIG. 2 indicate that the IM injection of epinephrine achieved a change in blood glucose levels of 15.5 mg/dL at 60 minutes and 1.57 mg/dL at 240 minutes, whereas the sublingual epinephrine tablet induced a change in blood glucose levels of 33.1 mg/dL at 60 minutes and 8.0 mg/dL at 240 minutes, an approximate 2.1-fold and 5.1-fold increase compared to the IM epinephrine injection, respectively. These results demonstrate that the sublingual epinephrine tablets induced an unexpected and prolonged increase in blood glucose levels indicative of a robust sympathetic response, which is required for anaphylactic rescue.

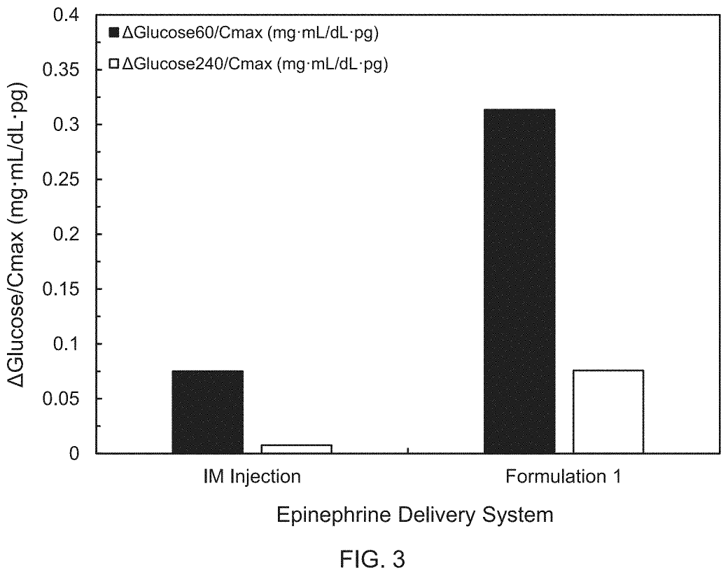

FIG. 3 depicts the temporal change in normalized serum glucose levels at 60 minutes (black) and 240 minutes (white) following the administration of the IM injection of epinephrine (“IM Injection”), described above, and the sublingual epinephrine tablet (“Formulation 1”), described above. Normalized serum glucose levels were obtained by dividing the serum glucose results of FIG. 2 by the maximum serum concentration results of FIG. 1. Because epinephrine is known to induce sympathetic effects and the delivery systems demonstrated significantly different levels of serum epinephrine, the change is serum glucose was normalized by the maximum epinephrine serum concentration to fully capture the magnitude of sympathetic activation elicited by the epinephrine delivery systems.

The results of FIG. 3 indicate that the IM injection of epinephrine achieved a change in normalized blood glucose levels of 0.075 mg mL/dL pg at 60 minutes and 0.008 mg mL/dL pg at 240 minutes, whereas the sublingual epinephrine tablet induced a change in blood glucose levels of 0.313 mg·mL/dL·pg at 60 minutes and 0.076 mg·mL/dL·pg at 240 minutes, an approximate 4.2-fold and 9.5-fold increase compared to the IM epinephrine injection, respectively. Notably, when accounting for serum epinephrine, these results further emphasize the outsized sympathetic response induced by the sublingual epinephrine tablets compared to epinephrine delivery systems of the prior art.

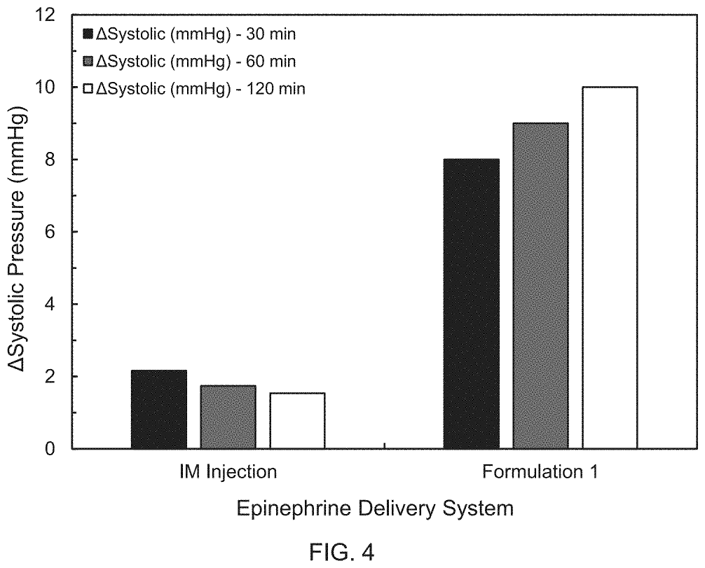

FIG. 4 depicts the temporal change in systolic blood pressure at 30 minutes (black), 60 minutes (grey), and 240 minutes (white) following the administration of the IM injection of epinephrine (“IM Injection”), described above, and the sublingual epinephrine tablet (“Formulation 1”), described above. The results of FIG. 4 indicate that the IM injection of epinephrine achieved a change in systolic blood pressure of 2.16 mmHg at 30 minutes, 1.74 mmHg at 60 minutes, and 1.54 mmHg at 120 minutes, whereas the sublingual epinephrine tablet induced a change in systolic blood pressure of 8 mmHg at 30 minutes, 9 mmHg at 60 minutes, and 10 mmHg at 120 minutes, an approximate 3.7-fold, 5.2-fold, and 6.5-fold increase compared to the IM epinephrine injection, respectively. These results demonstrate that the sublingual epinephrine tablets induced an unexpected and prolonged increase in systolic blood pressure levels indicative of a robust sympathetic response, which is required for anaphylactic rescue.

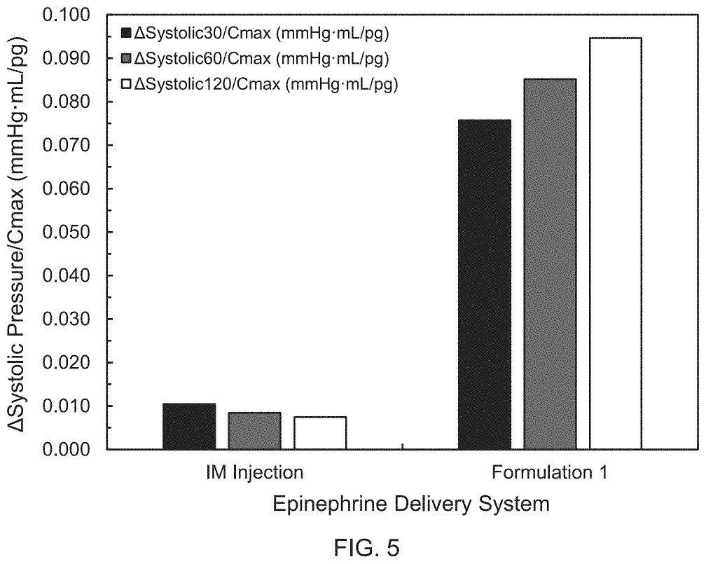

FIG. 5 depicts the temporal change in normalized systolic blood pressure at 30 minutes (black), 60 minutes (grey), and 240 minutes (white) following the administration of the IM injection of epinephrine (“IM Injection”), described above, and the sublingual epinephrine tablet (“Formulation 1”), described above. Normalized systolic blood pressure changes were obtained by dividing the systolic blood pressure results of FIG. 4 by the maximum serum concentration results of FIG. 1. Because epinephrine is known to induce sympathetic effects and the delivery systems demonstrated significantly different levels of serum epinephrine, the change is systolic blood pressure was normalized by the maximum epinephrine serum concentration to fully capture the magnitude of sympathetic activation elicited by the epinephrine delivery systems.

The results of FIG. 5 indicate that the IM injection of epinephrine achieved a change in normalized systolic blood pressure of 0.010 mmHg mL/pg at 30 minutes, 0.008 mmHg mL/pg at 60 minutes, and 0.007 mmHg mL/pg at 120 minutes, whereas the sublingual epinephrine tablet induced a change in systolic blood pressure of 0.076 mmHg·mL/pg at 30 minutes, 0.085 mmHg·mL/pg at 60 minutes, and 0.095 mmHg·mL/pg at 120 minutes, an approximate 7.6-fold, 10.6-fold, and 13.6-fold increase compared to the IM epinephrine injection, respectively. Notably, when accounting for serum epinephrine, these results further emphasize the outsized sympathetic response induced by the sublingual epinephrine tablets compared to epinephrine delivery systems of the prior art.

These are significant and unexpected findings. Serum epinephrine levels may be less useful in predicting the success of resolving anaphylaxis in a subject, because blood pressure drops when a subject experiences anaphylaxis. As blood pressure drops, blood flow is directed away from the extremities to the head and organs in the trunk. In this scenario, intramuscular injection of epinephrine may be insufficient because there is no mechanism that can transport the epinephrine from the site of injection to the systemic blood supply, likely due to decreased and/or inadequate blood pressure. Moreover, this effect may be further exacerbated for subjects that already have low blood pressure in the absence of anaphylaxis. Because the sublingual tablet, disclosed herein, does not rely on the same mechanism as intramuscular injection, i.e., high levels of serum epinephrine, but rather relies on sympathetic activation to elicit a response, the likelihood of success of resolving anaphylaxis is higher in the sublingual epinephrine tablets of the present disclosure compared to epinephrine delivery systems known in the prior art.

### Example 3

A sublingual epinephrine tablet (25 mg dose of L-epinephrine) (n=100), as described herein (“Formulation 2”), was evaluated in a clinical trial against an epinephrine auto-injector (“EpiPen”—Epinephrine Injection, USP Auto-Injector, 0.3 mg (Mylan Specialty L.P.)) (n=100).

Treatments above were administered by two means. Sublingual tablets were administered by placing the tablet under the subject's tongue, as far back as possible. The subject was instructed not to swallow, chew, suck, or bite the tablet. Complete dissolution of the tablet is confirmed by clinical staff. Second, EpiPen injections were administered by clinical staff to the anterolateral aspect of the thigh of the subject, in accordance with CRO-approved instructions.

The clinical trial enrollment, inclusion and exclusion criteria, and eligibility and continuation requirements were the same as those in Example 2.

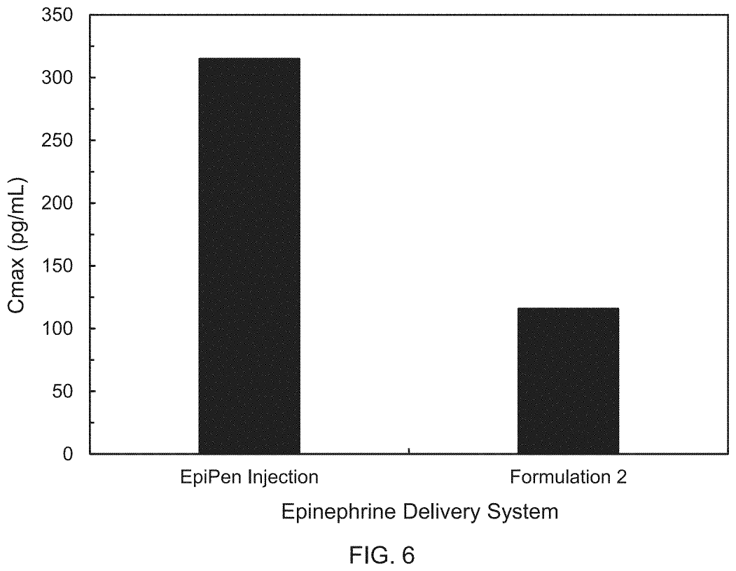

The clinical trial was performed to evaluate the maximum serum concentrations (Cmax) of epinephrine following the administration of the aforementioned treatments; and metrics associated with sympathetic response, which is required for anaphylactic rescue, namely: (i) change in serum glucose levels (at 3, 6, 10, 15, 20, 30, 45, 60, 90, 120, and 240 minutes following administration), (ii) change in systolic blood pressure (at the same time points), (iii) heart rate (at the same time points excluding 240 minutes), and (iv) incidence of gastrointestinal or other evidence of sympathetic activation, aside from change in blood glucose levels, systolic blood pressure, and heart rate. The data from the clinical studies are reported in FIGS. 6-12.

FIG. 6 depicts the observed Cmax following the administration of the EpiPen injection of epinephrine (“EpiPen Injection”), described above, and the sublingual epinephrine tablet (“Formulation 2”), described above. FIG. 6 demonstrates that the EpiPen injection produced a mean Cmax of about 315 pg/mL, which was significantly larger than mean Cmax of about 116 pg/mL observed for the sublingual epinephrine tablets of Formulation 2. This result was unexpected, as the sublingual epinephrine tablet was expected to have a similar or larger maximum serum concentration of epinephrine compared to an EpiPen injection.

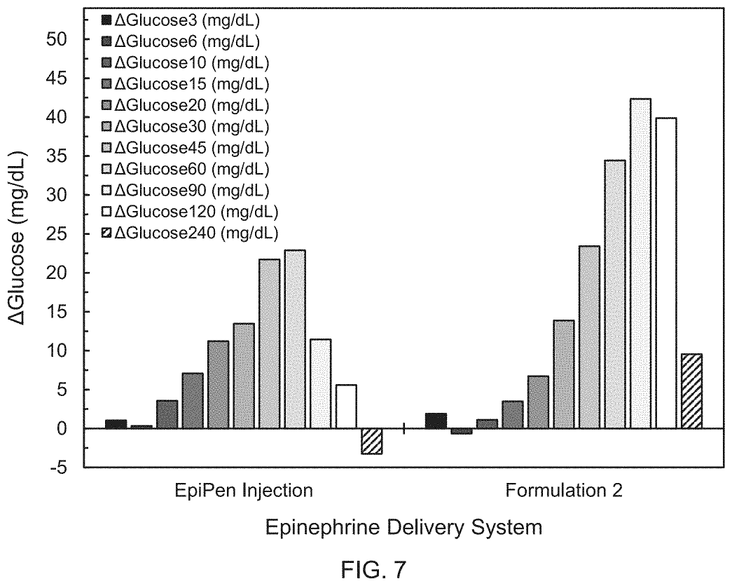

FIG. 7 depicts the temporal change in serum glucose levels following the administration of the EpiPen injection of epinephrine (“EpiPen Injection”), described above, and the sublingual epinephrine tablet (“Formulation 2”), described above. The results of FIG. 7 indicate that the EpiPen injection achieved a change in blood glucose levels of about 1.04 mg/dL at 3 minutes, 7.06 mg/dL at 15 minutes, 13.5 mg/dL at 30 minutes, 22.9 mg/dL at 60 minutes, and 5.59 mg/dL at 120 minutes, whereas the sublingual epinephrine tablet induced a change in blood glucose levels of about 1.89 mg/dL at 3 minutes, 3.49 mg/dL at 15 minutes, 13.9 mg/dL at 30 minutes, 34.4 mg/dL at 60 minutes, and 39.9 mg/dL at 120 minutes, an approximate 1.8-fold increase, 0.5-fold decrease, equal change, 1.5-fold increase, and 7.1-fold increase compared to the EpiPen injection, respectively. These results demonstrate that the sublingual epinephrine tablets induced a similar sympathetic response to the EpiPen injection at short time scales, and an unexpected and prolonged increase in blood glucose levels indicative of a robust sympathetic response, which is required for anaphylactic rescue, at large time scales.

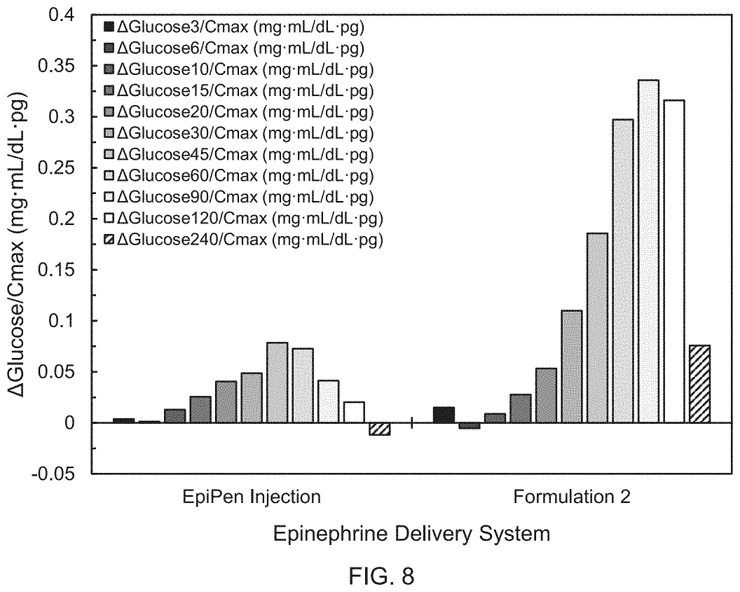

FIG. 8 depicts the temporal change in normalized serum glucose levels following the administration of the EpiPen injection of epinephrine (“EpiPen Injection”), described above, and the sublingual epinephrine tablet (“Formulation 2”), described above. Normalized serum glucose levels were obtained by dividing the serum glucose results of FIG. 7 by the maximum serum concentration results of FIG. 6. Because epinephrine is known to induce sympathetic effects and the delivery systems demonstrated significantly different levels of serum epinephrine, the change is serum glucose was normalized by the maximum epinephrine serum concentration to fully capture the magnitude of sympathetic activation elicited by the epinephrine delivery systems.

The results of FIG. 8 indicate that the EpiPen injection achieved a change in normalized blood glucose levels of about 0.003 mg·mL/dL·pg at 3 minutes, 0.026 mg·mL/dL·pg at 15 minutes, 0.049 mg·mL/dL·pg at 30 minutes, 0.073 mg·mL/dL·pg at 60 minutes, and 0.020 mg·mL/dL·pg at 120 minutes, whereas the sublingual epinephrine tablet induced a change in normalized blood glucose levels of about 0.015 mg·mL/dL·pg at 3 minutes, 0.028 mg·mL/dL·pg at 15 minutes, 0.11 mg·mL/dL·pg at 30 minutes, 0.30 mg·mL/dL·pg at 60 minutes, and 0.32 mg·mL/dL·pg at 120 minutes, an approximate 5-fold increase, 1.1-fold increase, 2.2-fold increase, 4.1-fold increase, and 16-fold increase compared to the EpiPen injection, respectively. These results demonstrate that the sublingual epinephrine tablets induced an unexpected and prolonged increase in blood glucose levels indicative of a robust sympathetic response, which is required for anaphylactic rescue.

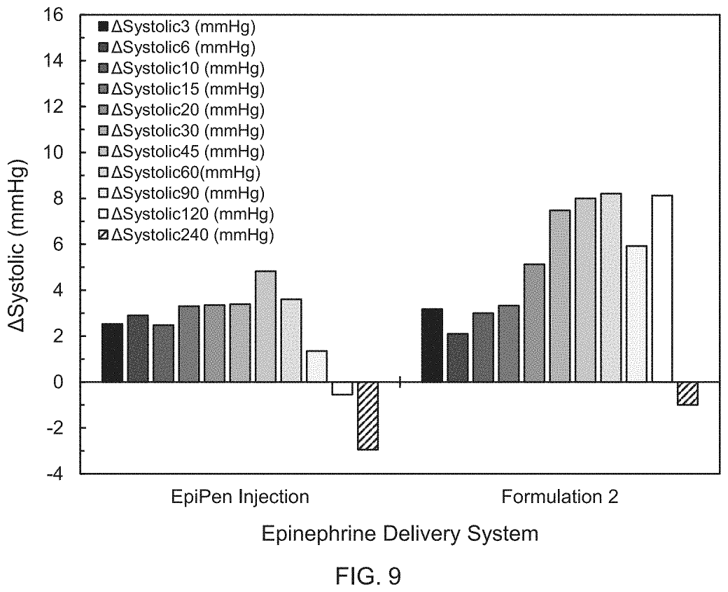

FIG. 9 depicts the temporal change in systolic blood pressure following the administration of the EpiPen injection of epinephrine (“EpiPen Injection”), described above, and the sublingual epinephrine tablet (“Formulation 2”), described above. The results of FIG. 9 indicate that the EpiPen injection achieved a change in systolic blood pressure of about 2.53 mmHg at 3 minutes, 3.30 mmHg at 15 minutes, 3.39 mmHg at 30 minutes, 3.60 mmHg at 60 minutes, and −0.55 mmHg at 120 minutes, whereas the sublingual epinephrine tablet induced a change in systolic blood pressure of about 3.18 mmHg at 3 minutes, 3.33 mmHg at 15 minutes, 7.48 mmHg at 30 minutes, 8.21 mmHg at 60 minutes, and 8.13 mmHg at 120 minutes, an approximate 1.3-fold increase, equal change, 2.2-fold increase, 2.3-fold increase, and 16.8-fold increase compared to the EpiPen injection, respectively. These results demonstrate that the sublingual epinephrine tablets induced a similar sympathetic response to the Epi-Pen injection at short time scales, and an unexpected and prolonged increase in systolic blood pressure indicative of a robust sympathetic response, which is required for anaphylactic rescue, at large time scales.

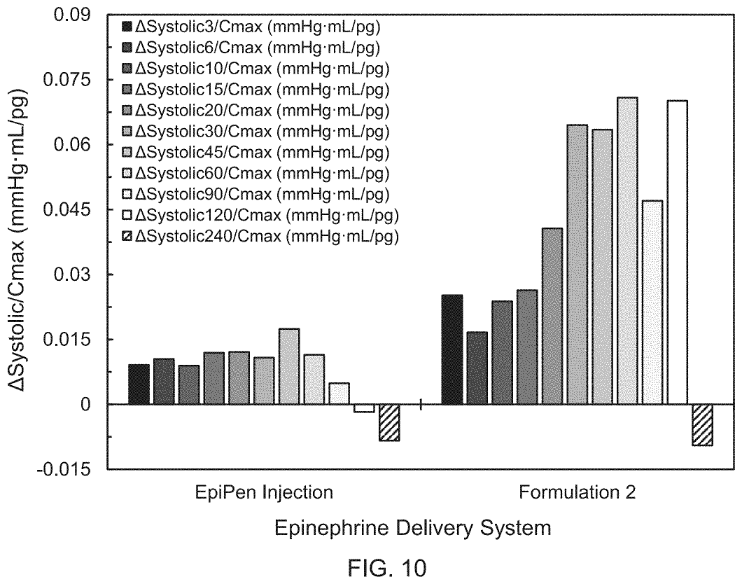

FIG. 10 depicts the temporal change in normalized systolic blood pressure following the administration of the EpiPen injection of epinephrine (“EpiPen Injection”), described above, and the sublingual epinephrine tablet (“Formulation 2”), described above. Normalized systolic blood pressure changes were obtained by dividing the systolic blood pressure results of FIG. 9 by the maximum serum concentration results of FIG. 6. Because epinephrine is known to induce sympathetic effects and the delivery systems demonstrated significantly different levels of serum epinephrine, the change is systolic blood pressure was normalized by the maximum epinephrine serum concentration to fully capture the magnitude of sympathetic activation elicited by the epinephrine delivery systems.

The results of FIG. 10 indicate that the EpiPen injection achieved a change in normalized systolic blood pressure of about 0.009 mmHg·mL/pg at 3 minutes, 0.012 mmHg·mL/pg at 15 minutes, 0.011 mmHg·mL/pg at 30 minutes, 0.011 mmHg·mL/pg at 60 minutes, and −0.002 mmHg·mL/pg at 120 minutes, whereas the sublingual epinephrine tablet induced a change in normalized systolic blood pressure of about 0.025 mmHg·mL/pg at 3 minutes, 0.026 mmHg mL/pg at 15 minutes, 0.065 mmHg mL/pg at 30 minutes, 0.071 mmHg mL/pg at 60 minutes, and 0.070 mmHg·mL/pg at 120 minutes, an approximate 2.8-fold increase, 2.2-fold increase, 5.9-fold increase, 6.5-fold increase, and 37-fold increase compared to the EpiPen injection, respectively. These results demonstrate that the sublingual epinephrine tablets induced an unexpected and prolonged increase in systolic blood pressure indicative of a robust sympathetic response, which is required for anaphylactic rescue.

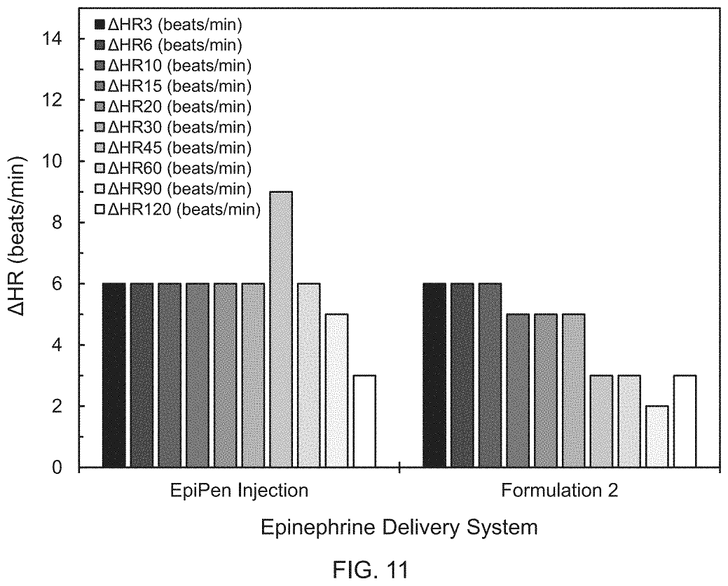

FIG. 11 depicts the temporal change in heart rate following the administration of the EpiPen injection of epinephrine (“EpiPen Injection”), described above, and the sublingual epinephrine tablet (“Formulation 2”), described above. The results of FIG. 11 indicate that the EpiPen injection achieved a change in heart rate of about 6 beats/min at 3 minutes, 6 beats/min at 15 minutes, 6 beats/min at 30 minutes, 6 beats/min at 60 minutes, and 5 beats/min at 120 minutes, whereas the sublingual epinephrine tablet induced a change in heart rate of about 6 beats/min at 3 minutes, 5 beats/min at 15 minutes, 5 beats/min at 30 minutes, 3 beats/min at 60 minutes, and 2 beats/min at 120 minutes, These results demonstrate that the sublingual epinephrine tablets induced a similar sympathetic response to the EpiPen injection at all time scales.

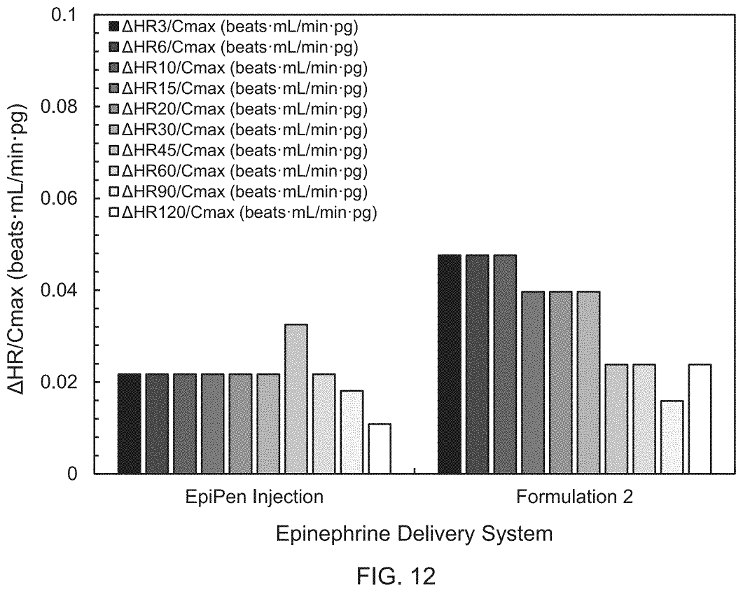

FIG. 12 depicts the temporal change in normalized heart rate following the administration of the EpiPen injection of epinephrine (“EpiPen Injection”), described above, and the sublingual epinephrine tablet (“Formulation 2”), described above. Normalized heart rate changes were obtained by dividing the heart rate results of FIG. 11 by the maximum serum concentration results of FIG. 6. Because epinephrine is known to induce sympathetic effects and the delivery systems demonstrated significantly different levels of serum epinephrine, the change in heart rate was normalized by the maximum epinephrine serum concentration to fully capture the magnitude of sympathetic activation elicited by the epinephrine delivery systems.

The results of FIG. 12 indicate that the EpiPen injection achieved a change in normalized heart rate of about 0.022 beats·mL/min·pg at 3 minutes, 0.022 beats·mL/min·pg at 15 minutes, 0.022 beats·mL/min·pg at 30 minutes, 0.022 beats·mL/min·pg at 60 minutes, and 0.018 beats mL/min pg at 120 minutes, whereas the sublingual epinephrine tablet induced a change in normalized heart rate of about 0.048 beats·mL/min·pg at 3 minutes, 0.040 beats·mL/min·pg at 15 minutes, 0.040 beats·mL/min·pg at 30 minutes, 0.024 beats·mL/min·pg at 60 minutes, 0.016 beats·mL/min·pg at 120 minutes, an approximate 2.2-fold increase, 1.8-fold increase, 1.8-fold increase, 1.1-fold increase, and 0.9-fold decrease compared to the EpiPen injection, respectively. These results demonstrate that the sublingual epinephrine tablets induced an unexpected and prolonged increase in heart rate indicative of a robust sympathetic response, which is required for anaphylactic rescue.

These are significant and unexpected findings. Serum epinephrine levels may be less useful in predicting the success of resolving anaphylaxis in a subject, because blood pressure drops when a subject experiences anaphylaxis. As blood pressure drops, blood flow is directed away from the extremities to the head and organs in the trunk. In this scenario, intramuscular injection of epinephrine may be insufficient because there is no mechanism that can transport the epinephrine from the site of injection to the systemic blood supply, likely due to decreased and/or inadequate blood pressure. Moreover, this effect may be further exacerbated for subjects that already have low blood pressure in the absence of anaphylaxis. Because the sublingual tablet, disclosed herein, does not rely on the same mechanism as intramuscular injection, i.e., high levels of serum epinephrine, but rather relies on sympathetic activation to elicit a response, the likelihood of success of resolving anaphylaxis is higher in the sublingual epinephrine tablets of the present disclosure compared to epinephrine delivery systems known in the prior art.

1. A method of activating the sympathetic nervous system of a subject, the method comprising:
administering an epinephrine composition to the subject, the composition comprising:
an effective amount of epinephrine or a salt thereof;
an effective amount of citric acid or a salt thereof; and
one or more excipients;
wherein the activating the sympathetic nervous system is characterized by at least one physiological change in the subject selected from the group consisting of an increase in serum blood glucose levels, an increase in systolic blood pressure, an increased heart rate, and any combination thereof.

2. The method of claim 1, wherein the epinephrine is provided as L-epinephrine bitartrate.

3. The method of claim 2, wherein the epinephrine composition is administered to the subject via a route selected from the group consisting of sublingual, sublabial, and buccal.

4. The method of claim 3, wherein the increase in serum blood glucose levels are observed for at least about 60 minutes following administration of the epinephrine composition.

5. The method of claim 4, wherein the increase in serum blood glucose levels are observed for at least about 240 minutes following administration of the epinephrine composition.

6. The method of claim 3, wherein the increase in systolic blood pressure are observed for at least about 30 minutes following administration of the epinephrine composition.

7. The method of claim 6, wherein the increase in systolic blood pressure are observed for at least about 120 minutes following administration of the epinephrine composition.

8. The method of claim 3, wherein the effective amount of epinephrine is about 1 mg to about 50 mg.

9. The method of claim 8, wherein the effective amount of citric acid is about 0.5 mg to about 5.0 mg.

10. The method of claim 9, wherein the effective amount of epinephrine is about 25 mg, and the effective amount of citric acid is about 2.81 mg.

11. The method of claim 9, wherein the effective amount of epinephrine is about 25 mg, and the effective amount of citric acid is 4.0 mg.

12. An epinephrine composition comprising:
an effective amount of epinephrine or a salt thereof;
an effective amount of citric acid or a salt thereof; and
one or more excipients.

13. The epinephrine composition of claim 12, wherein the epinephrine is provided as L-epinephrine bitartrate.

14. The epinephrine composition of claim 13, wherein the epinephrine composition is formulated for administration via a route selected from the group consisting of sublingual, sublabial, and buccal.

15. The epinephrine composition of claim 14, wherein the effective amount of epinephrine is about 1 mg to about 50 mg.

16. The epinephrine composition of claim 15, wherein the effective amount of citric acid is about 0.5 mg to about 5.0 mg.

17. The epinephrine composition of claim 16, wherein the effective amount of epinephrine is about 25 mg, and the effective amount of citric acid is about 2.81 mg.

18. The epinephrine composition of claim 16, wherein the effective amount of epinephrine is about 25 mg, and the effective amount of citric acid is about 4.0 mg.

19. A method of treating or ameliorating anaphylaxis or a symptom thereof in a subject, the method comprising:
identifying that the subject is experiencing anaphylaxis or a symptom thereof;
administering an epinephrine composition to the subject, the composition comprising:
an effective amount of epinephrine or a salt thereof;
an effective amount of citric acid or a salt thereof; and
one or more excipients.

20. The method of claim 19, wherein the epinephrine is provided as L-epinephrine bitartrate.

21. The method of claim 20, wherein the epinephrine composition is administered to the subject via a route selected from the group consisting of sublingual, sublabial, and buccal.

22. The method of claim 21, wherein the effective amount of epinephrine is about 1 mg to about 50 mg.

23. The method of claim 22, wherein the effective amount of citric acid is about 0.5 mg to about 5.0 mg.

24. The method of claim 23, wherein the effective amount of epinephrine is about 25 mg, and the effective amount of citric acid is about 2.81 mg.

25. The method of claim 24, wherein the effective amount of epinephrine is about 25 mg, and the effective amount of citric acid is 4.0 mg.

26. The method of claim 21, wherein the anaphylaxis or symptom thereof is caused by allergen immunotherapy.
